(19) United States
(12) Patent Application Publication
(10) Pub. No.: US 2005/0115855 A1
(43) Pub. Date: Jun. 2, 2005

(54) Feminine hygiene kit

(76) Inventors: Ronit Hilel, Modii'n (IL); Yuval Hilel, Modii'n (IL)

Correspondence Address: DR. MARK FRIEDMAN LTD.;C/o Bill Polkinghorn, Discovery Dispatch, 9003 Florin Way, Upper Marlboro, MD, 20772, US

(21) Appl. No.: 10/725,006

(22) Filed: Dec. 2, 2003

(51) Int. Cl. A61L 15/00

(52) U.S. Cl. 206/440; 206/581

(57) ABSTRACT

The preferred embodiment of a container of the present invention, as illustrated herein, is a case with a first, or main, compartment that is configured to hold both tampons and feminine pads. The case also has a second compartment, which may be used for smaller items such as, but not limited to, medications, that is accessible without opening the main compartment. The region of the case that houses each of the different shaped items is molded to accommodate the items. That is, the region that holds the tampons extends away from the rest of case and has a rounded oval shape. The overall cross sectional contour may be flared to accommodate the shape of some brands of mini-pads. The case is designed with smooth curves for functionality, so as to slide smoothly and easily into and out of a purse or briefcase, for example, as well as for the atheistic value.

### FIELD AND BACKGROUND OF THE INVENTION

The present invention relates to containers for storing feminine hygiene articles and, in particular, it concerns a compartmentalized container.

Containers for the storage and transportation of feminine hygiene articles for personal use are known. They have become popular since women are spending more time away from home and find it convenient to carry a small stock of their favorite hygiene articles with them. These often include a combination of all three articles; tampons, feminine pads, and prescription or over-the-counter medications. Many women, while maintaining their privacy as to their menstrual state, enjoy carrying accessories that are sleek and aesthetically please and may also be colorful.

The container disclosed in U.S. Pat. No. 5,004,106, to Blumstock et al., discloses a multi-compartment container having a large compartment for tampons and a smaller compartment for such items as pills for example. Although the tampons are stored in the container, no mechanism is provided to secure them in place thereby protecting them from damage or to keep them in the container should the container accidentally open. In variant embodiments, Blumstock et al. describes addition compartments each with its own lid. Therefore, the user must open a different lid for each article. For situations where both a tampon and a pad are desired, two separate compartments must be opened.

There is therefore a need for a container for storing feminine hygiene articles that holds tampons securely in place and which holds two similar articles in separate regions of the same compartment. It would be of further benefit if the outer contour of the container were configured with smooth curves.

### SUMMARY OF THE INVENTION

The present invention is a container for transporting feminine hygiene articles that holds tampons securely in place.

According to the teachings of the present invention there is provided, a container for storage of feminine-hygiene articles, the container comprising: (a) an integrally formed case configured as two case segments interconnected by an integrally formed hinge, so as to be deployable between an open and a closed state, such that in the closed state the case encloses a first storage compartment, the first storage compartment configured so as to house at least two different articles each having a different maximum length such that a first article is stored in a first region of the first storage compartment and a second article is stored in a second region of the first storage compartment, and a longitudinal cross sectional length of the first region is different than a of a longitudinal cross sectional length of the second region; and (b) a retaining element deployed within the first storage compartment, the retaining element configured so as to hold at least one feminine hygiene article.

According to a further teaching of the present invention, the retaining element includes at least one pair of resilient grasping extensions configured to grasp a cylindrical article between them.

According to a further teaching of the present invention, one of the case segments further includes at least two through bores each of having a different shape, and the retaining element includes at least two complementary attachment projection configured so as to be insertable into and substantially fill the at least two through bores, thereby securing and providing proper alignment of the retaining element within the case.

According to a further teaching of the present invention, one of the case segments further includes at least one through bore in the shape of an alphanumeric character, and the retaining element includes at least one complementary attachment projection configured so as to be insertable into and substantially fill the at least one through bore, thereby securing and providing proper alignment of the retaining element within the case.

According to a further teaching of the present invention, there is also provided, at least one of the case segments configured with at least a second storage compartment that is accessible from outside the container, and at least one storage compartment cover connected to the case so as to close the at least a second storage compartment.

According to a further teaching of the present invention, there is also provided, a false floor deployed within the first storage compartment and attached to the at least one of the case segments that includes the second storage compartment, so as to substantially cover topographical differences in an inner surface of the case created by the second compartment, thereby providing a substantially flat compartment floor.

According to a further teaching of the present invention, each of the case segments further includes a sealing rim such that in the closed state each the sealing rim contacts the other the sealing rim thereby sealing the case.

According to a further teaching of the present invention, each of the case segments includes complementary portions of an integrally formed latch configured to releasably retain the case in the closed state.

### BRIEF DESCRIPTION OF THE DRAWINGS

The invention is herein described, by way of example only, with reference to the accompanying drawings, wherein:

FIG. 1 is an isometric front view of a container constructed and operable according to the teachings of the present invention, shown here in an open state illustrating placement of feminine articles;

FIGS. 2 and 3 are isometric side views of the container of FIG. 1;

FIG. 4 is an isometric front view of the container of FIG. 1, shown without the feminine articles or the false floor;

FIG. 5 is an isometric view of a retaining element constructed and operable according to the teachings of the present invention, showing the side of the retaining element configured with grasping extensions;

FIG. 6 is a side view of the retaining element of FIG. 5;

FIG. 7 is a side view of the retaining element of FIG. 5, shown with a tampon secured for storage;

FIG. 8 is an isometric top view of the container of FIG. 1 showing the cut out letters;

FIG. 9 is an isometric view of a retaining element of FIG. 5, showing the side of the retaining element configured with complementary attachment projections in the form of letters;

FIG. 10 is a cross-sectional detail showing the engagement of the complementary attachment projections of FIG. 9 inserted into the cut out letters of FIG. 8;

FIG. 11 is an isometric view of the container of FIG. 1 showing the second compartment;

FIG. 12 is an isometric view of a false floor constructed and operable according to the teachings of the present invention; and

FIG. 13 is an isometric view of the container of FIG. 1 with the false floor in place.

### DESCRIPTION OF THE PREFERRED EMBODIMENTS

The present invention is a container for transporting feminine hygiene articles that holds tampons securely in place.

The principles and operation of a container for transporting feminine hygiene articles according to the present invention may be better understood with reference to the drawings and the accompanying description.

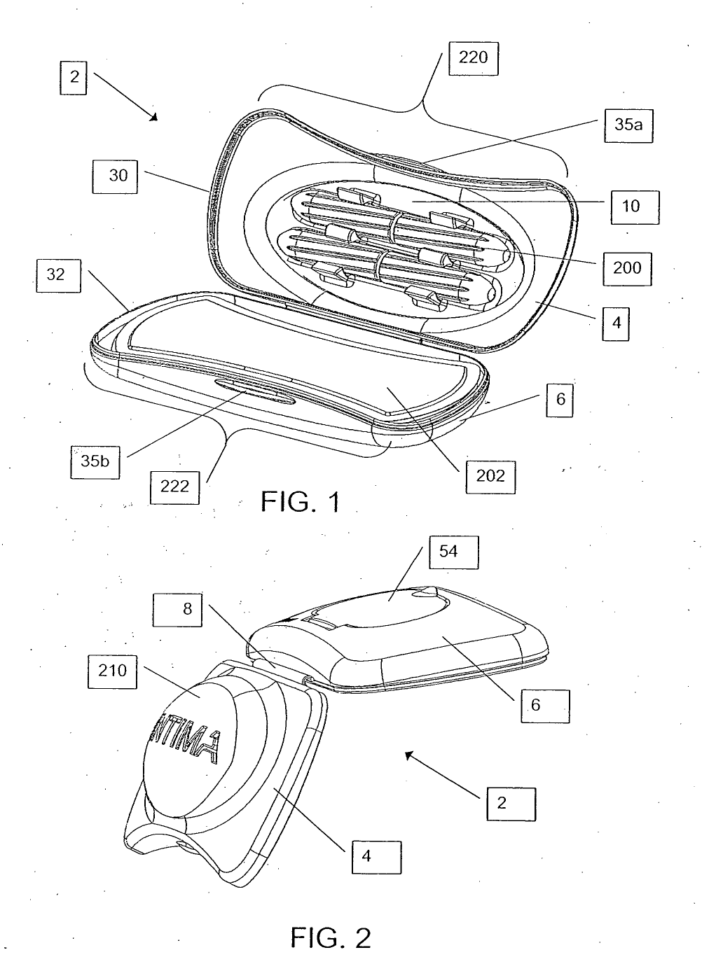

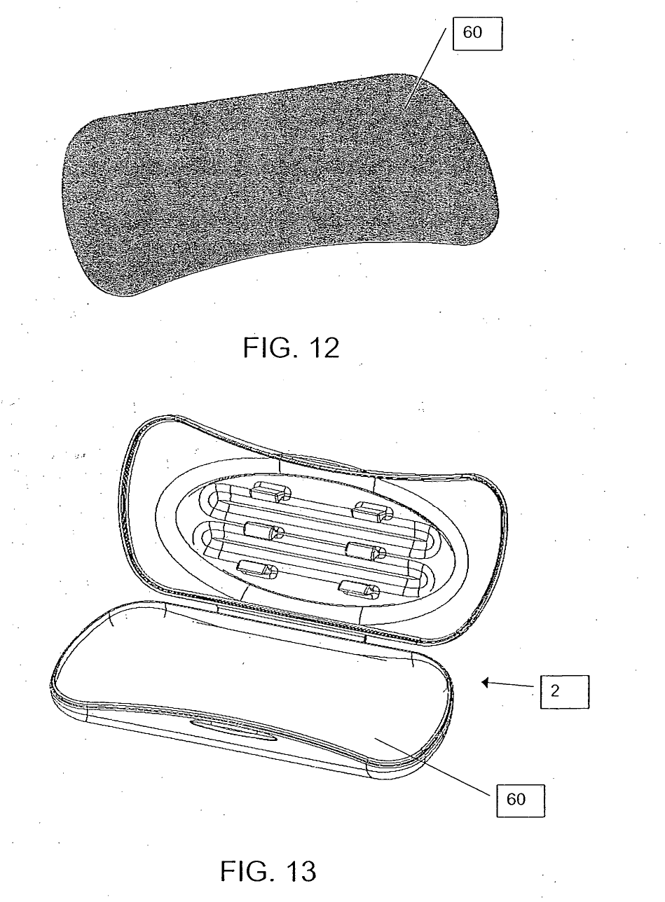

By way of introduction, the preferred embodiment of a container of the present invention, as illustrated in FIG. 1, includes a case 2 with a first, or main, compartment that is configured to hold both tampons 200 and feminine pads 202. The case 2 also has a second compartment 50, as seen in FIG. 11, which may be used for smaller items such as, but not limited to, pills. As is clearly illustrated in FIGS. 2, 4 and 8, the regions of the case that house each of the different shaped items is molded to accommodate the items. That is, region 210 of the first compartment houses the retaining element 10, which holds the tampons, and therefore extends away from the rest of case segment 4 as a rounded oval, for example. Region 212 on the other hand houses pads, which have a longer maximum length that the tampons housed in region 210. Therefore, the longitudinal cross-sectional length 212a of region 212 is longer than the longitudinal cross-sectional length 210a of region 210 (see FIG. 10). Although not shown, it will be appreciated form the drawing that the latitudinal cross-sectional lengths of regions 210 and 212 are also different. Regions 220 and 222 of the case (seen with more clarity in FIG. 12) may be flared to accommodate the shape of some brands of mini-pads. The outer contour of the case is designed with smooth curves for functionality, so as to slide smoothly and easily into and out of a purse or briefcase, for example, as well as for atheistic value.

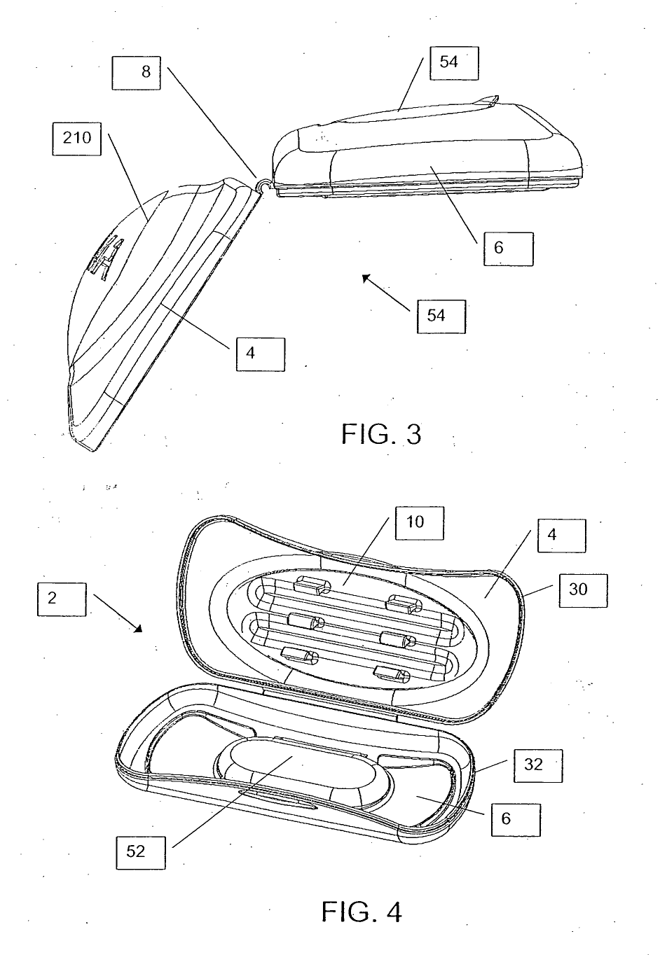

Referring now to a preferred embodiment of the present invention and the drawings, FIGS. 2 and 3 illustrate the one-piece integrally formed case 2, which has two segments 4 and 6 that are interconnected by the integrally formed hinge 8. These Figures also offer a side view of region 210 in case segment 4 and the cover 54 that encloses the second compartment 50.

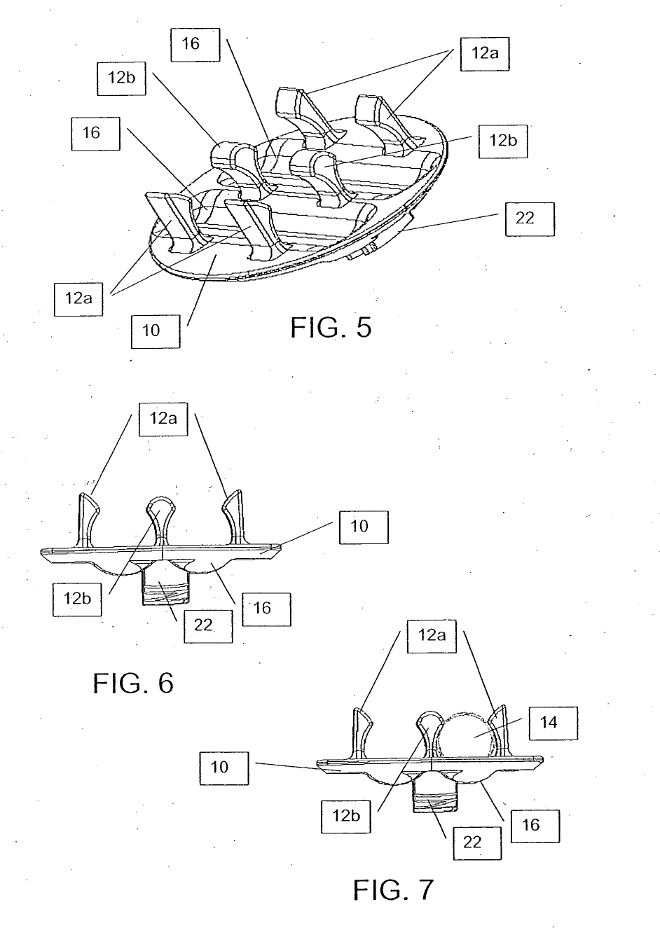

FIG. 4 provides a clear view of the retaining element 10 that is deployed in region 210 of case segment 4. Also visible is the back 52 of the second compartment 50 projecting into case segment 6. In this embodiment, the retaining element is configured with four “pairs” of resilient grasping extensions 12. That is, two outer grasping extensions 12a are aligned on opposite sides of a center grasping extension 12b such that one outer grasping 12a extension and a corresponding portion of the center grasping extension 12b form a “pair.” See also FIGS. 5, 6 and 7. FIG. 7 includes a tampon 14 deploy for storage between two grasping extensions 12a and 12b. As illustrated herein, the retaining element is further configured with trough regions 16, which provide further stability to the article being held between the grasping extensions 12a and 12b. It should be noted that the grasping extensions are preferably configured for grasping articles with a substantially circular cross-section; however, the grasping extensions may be of substantially any configuration that will grasp the intended article. Further, the grasping extension may be deployed in substantially any suitable arrangement. The example illustrated here is that of two parallel rows. It will be readily appreciated that the size or shape, or both, of region 210, may be modified so as to accept a retaining element 10 of substantially any shape or size, such as but not limited to, a retaining element 10 for tampons of shapes different from those illustrated herein. A further alternative may include configuration of the retaining element 10 so as to hold any number (e.g. 1-10) of tampons. Retaining element 10 may also be configured to hold any other article, or any combination of articles. It should be noted that the grasping extensions may be configured to hold substantially any article whose size and shape are suitable for storage within the case.

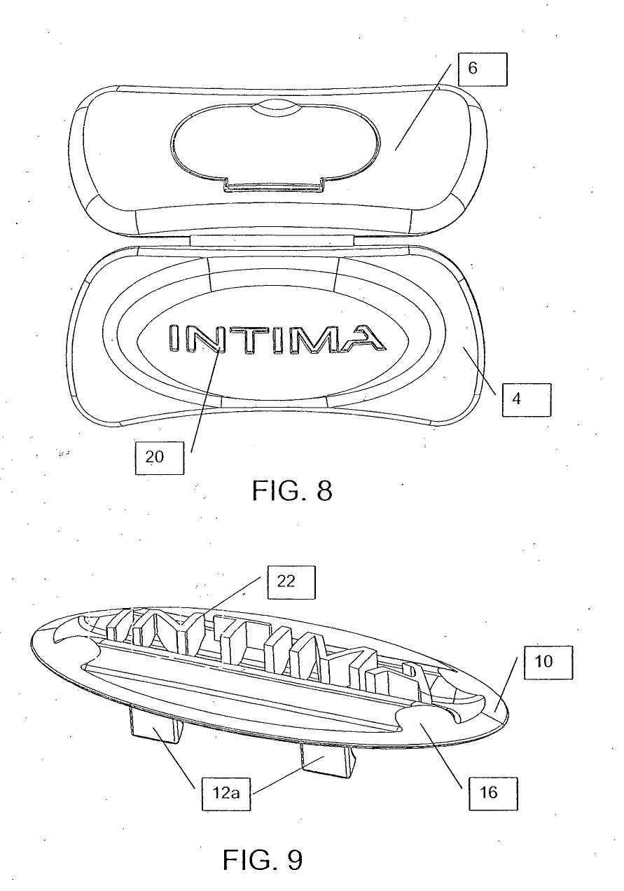

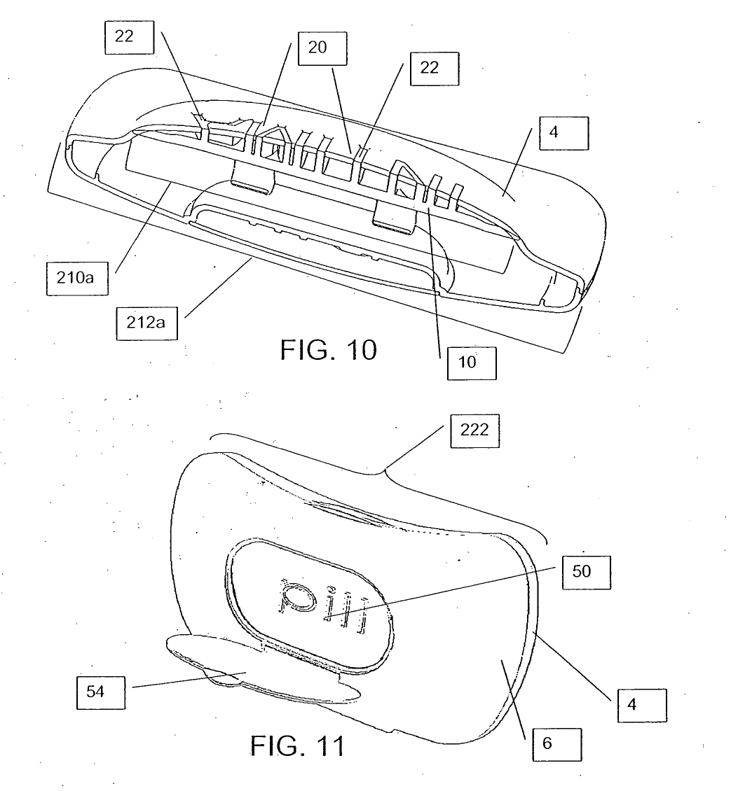

Case segment 4 may also be configured with a through bore, that is, an area in which the material of the case is cut-away. In the illustration of FIG. 8, the through bore is implemented as a plurality of through bores 20, each of which is in the form of a letter, such that they spell the word “INTIMA.” It will be readily appreciated that the through bore may be configured in substantially any size and graphic form such as, but not limited to, alphanumeric characters, geometric shapes, logos, and insignias. The retaining element 10 is configured with a complementary attachment projection 22 configured so as to be insertable into and substantially fill the plurality of through bores 20, thereby securing and providing proper alignment for the retaining element 10 within said case 2, as illustrated in FIG. 10. It will be readily appreciated that the color of the case 2 and the color of the complementary attachment projection 22 may be different so as to achieve a desired visual effect. Alternatively, the retaining element 10 may be secured within the case 2 by any appropriate method known in the art.

As illustrated in FIG. 11, case segment 6 may be configured with a second storage compartment 50 that is accessible from outside the container 2, that is, without opening the main compartment. The storage compartment cover 54 is hingedly connected to the case so as to enclose the second storage compartment 50.

Due to the topographical differences in the inner surface of case segment 6 created by the projection of the second compartment 50 into the volume of the first compartment, a false floor 60 may be inserted into case segment 6, thereby providing a substantially flat compartment floor.

Each of the case segments 4 and 6 further includes a sealing rim 30 and 32 such that in the closed state each of the sealing rims 30 and 32 contacts the other sealing rim thereby sealing the case 2. In the preferred embodiment illustrated herein, the sealing rims 30 and 32 are configured so as to mutually engage each other along substantially the entirety of their length. Regions 35a and 35b are provided to facilitate opening of the case 2. Alternatively, an integrally formed latch may be configured to releasably retain the case 2 in a closed state. The latch may be form as part of the sealing rims 30 and 32 or as a separate element.

It will be appreciated that the above descriptions are intended only to serve as examples, and that many other embodiments are possible within the spirit and the scope of the present invention.

1. A container for storage of feminine-hygiene articles, the container comprising:
(a) an integrally formed case configured as two case segments interconnected by an integrally formed hinge, so as to be deployable between an open and a closed state, such that in said closed state said case encloses a first storage compartment, said first storage compartment configured so as to house at least two different articles each having a different maximum length such that a first article is stored in a first region of said first storage compartment and a second article is stored in a second region of said first storage compartment, and a longitudinal cross sectional length of said first region is different than a of a longitudinal cross sectional length of said second region; and
(b) a retaining element deployed within said first storage compartment, said retaining element configured so as to hold at least one feminine hygiene article.

2. The container of claim 1, wherein said retaining element includes at least one pair of resilient grasping extensions configured to grasp a cylindrical article between them.

3. The container of claim 1, wherein one of said case segments further includes at least two through bores each of having a different shape, and said retaining element includes at least two complementary attachment projection configured so as to be insertable into and substantially fill said at least two through bores, thereby securing and providing proper alignment of said retaining element within said case.

4. The container of claim 3, wherein one of said case segments further includes at least one through bore in the shape of an alphanumeric character, and said retaining element includes at least one complementary attachment projection configured so as to be insertable into and substantially fill said at least one through bore, thereby securing and providing proper alignment of said retaining element within said case.

5. The container of claim 1, further comprising at least one of said case segments configured with at least a second storage compartment that is accessible from outside the container, and at least one storage compartment cover connected to said case so as to close said at least a second storage compartment.

6. The container of claim 5, further including a false floor deployed within said first storage compartment and attached to said at least one of said case segments that includes said second storage compartment, so as to substantially cover topographical differences in an inner surface of said case created by said second compartment, thereby providing a substantially flat compartment floor.

7. The container of claim 1, wherein each of said case segments further includes a sealing rim such that in said closed state each said sealing rim contacts the other said sealing rim thereby sealing said case.

8. The container of claim 1, wherein each of said case segments includes complementary portions of an integrally formed latch configured to releasably retain said case in said closed state.
